(19) United States
(12) Patent Application Publication
(10) Pub. No.: US 2018/0169149 A1
(43) Pub. Date: Jun. 21, 2018

(54) OXYGENATED DEMINERALIZED BONE MATRIX FOR USE IN BONE GROWTH

(71) Applicant: THERACELL, INC., Northridge, CA (US)

(72) Inventors: Frank M. Phillips, Highland Park, IL (US); Stephen H. Hochschuler, Paradise Valley, AZ (US); Dan Gazit, Maccabim (IL); Gadi Pelled, Rishon-LeZion (IL); Zulma Gazit, Maccabim (IL)

(73) Assignee: THERACELL, INC.

(21) Appl. No.: 15/898,138

(22) Filed: Feb. 15, 2018

Related U.S. Application Data
(62) Division of application No. 13/200,961, filed on Oct. 4, 2011.
(60) Provisional application No. 61/389,875, filed on Oct. 5, 2010.
(60) Provisional application No. 61/436,438, filed on Jan. 26, 2011.

(51) Int. Cl. A61K 35/32; A61K 9/00; A61L 27/36

(52) U.S. Cl. CPC A61K 35/32; A61L 2430/02; A61L 27/3608; A61K 9/0026

(57) ABSTRACT

An improved composition for inducing bone growth is provided that is a combination of at least DBM and an oxygen carrier. Injection/implantation of a composition of DBM and an oxygen carrier (e.g. a perfluorocarbon) results in enhancement of bone formation compared to DBM alone.

### CROSS-REFERENCE TO RELATED APPLICATION(S)

This is a divisional application of U.S. patent application Ser. No. 13/200,961, filed Oct. 4, 2011, which claims priority to and the benefit of U.S. Provisional Application Ser. Nos. 61/389,875, filed on Oct. 5, 2010, and 61/436,438 filed on Jan. 26, 2011, the entire contents of both of which are incorporated herein by reference.

### BACKGROUND

A rapid and effective method for inducing bone formation has long been a need in the field of orthopedic and plastic surgery. The ability of bone to heal and of fusions to form is based on three key concepts: osteogenesis, osteoinduction, and osteoconduction.

Osteogenesis, defined as the ability to produce new bone, is determined by the presence of osteoprogenitor cells and osteogenic precursor cells in the area. Both fresh autografts and bone marrow cells contain osteogenic cells, although often in decreased numbers in the elderly patient (Heim G A, Dayoub H, and Jane J A Jr, Neurosurg Focus, 10(4), E5, 2001). Osteoconductive properties are determined by the presence of a scaffold that allows for vascular and cellular migration, attachment, and distribution (Heim G A, Dayoub H, and Jane J A Jr, Neurosurg Focus, 10(4), E4, 2001). Osteoconduction may be achieved through the use of autografts, allografts, DBM (demineralized bone matrix), hydroxyapatite, and collagen. Osteoconductive properties may be altered by structure, pore size, and porosity of the scaffold (Helm et al., Neurosurg Focus, 10(4), E4, 2001). Osteoinduction is defined as the ability to stimulate stem cells to differentiate into mature bone forming cells through stimulation by local growth factors (Subach B R, Haid R W, Rodts G E, et al., Neurosurg Focus, 10(4): Article 3, 2001). Bone morphogenetic proteins and DBM are the most potent osteoinductive materials, although alto- and autografts have some osteoinductive properties (Kalfas I H, Neurosurg Focus 10(4), E1, 2001).

Synthetic and natural materials have become used as scaffolds or adjuncts to scaffolds for conditions requiring bone formation such as spinal fusion (e.g., U.S. Patent Application Publication No. 2009/0214649). These materials may include extracellular matrices, DBMs, polymers, and ceramics. The goal of using these scaffolds is to induce osteogenesis through osteoconduction and to provide a delivery system for osteoinductive agents. Extracellular matrices such as collagen and glycosaminoglycans are able to aid in the differentiation of osteoprogenitor cells and bind osteogenic growth factors (Helm et al., Neurosurg Focus, 10(4): E4, 2001). Furthermore, the chemical and mechanical properties of these matrices may be altered depending on theft potential use. The use of demineralized bone matrix (DBM) in spinal fusion has been studied in both animals and humans. Although initial fusion success has been demonstrated in animals, studies in humans have shown autologous bone to produce higher fusion rates (Jorgenson S S, Lowe T G, France J, et al., Spine, 19:2048-2053, 1994). Polymers, such as poly-glycolic acid, poly-L-lactic acid, and polylactic-co-glycolic acid, have been used in clinical studies (Helm et al., Neurosurg Focus, 10(4): E4, 2001). These materials are osteoconductive and are able to deliver osteoinductive factors, but their efficacy is hindered by foreign-body reactions and by mild toxicities produced during biodegradation. Accordingly, further refinement is needed to develop an osteoconductive and osteoinductive DBM composition for bone growth and repair, that is easily implemented, and does not require the culturing of cells.

### SUMMARY

In one embodiment of the present invention, a composition for inducing bone growth is provided, the composition includes an oxygen carrier and demineralized bone matrix (DBM).

In a second embodiment of the present invention, the oxygen carrier is a perfluorocarbon.

In a third embodiment of the present invention, a method of inducing bone growth is provided, the method including combining an oxygen carrier and DBM to form a mixture, and implanting an effective amount of the mixture into a subject.

### BRIEF DESCRIPTION OF THE DRAWINGS

The patent or application file contains at least one drawing executed in color. Copies of this patent or patent application publication with color drawings will be provided by the Office upon request and payment of the necessary fee.

These and other features and advantages of the present invention will be better understood by reference to the following detailed description when considered in conjunction with the accompanying drawings, wherein:

FIGS. 1A-1D show micro computated tomography (micro-CT) images of bone growth in mice 21 days after implantation; 1A) DBM in PBS (2D analysis); 1B) DBM in PBS (3D analysis); 1C) DBM+PFTBA (2D analysis); 1D) DBM+PFTBA (3D analysis);

FIG. 2 is a histogram depicting bone volume measured from the micro-CT images;

FIGS. 3A-3B show histological analysis of bone growth in mice 21 days after implantation of 3A) DBM in PBS; 3B) DBM and PFTBA; (endochondral bone formation is outlined in yellow);

FIGS. 4A-4B show histological analysis of bone growth in mice 21 days after implantation of 4A) DBM in PBS; 4B) DBM and PFTBA; (EBF=endochondral bone formation);

FIGS. 5A-5B show histological analysis of bone growth in mice 21 days after implantation of 5A) DBM in PBS; 5B) DBM and PFTBA;

FIGS. 6A-6B show histological analysis of bone growth in mice 21 days after implantation of 6A) DBM in PBS; 6B) DBM and PFTBA;

FIGS. 7A-7F show micro computated tomography (micro-CT) images of bone growth in mice 21 days after implantation; 7A) DBM and bone chips in PBS (2D analysis); 7B) DBM and bone chips in PBS (segmented analysis); 7C) DBM and bone chips in PBS (3D analysis); 7D) DBM and bone chips in PFTBA (2D analysis); 7E) DBM and bone chips in PFTBA (segmented analysis); 7F) DBM and bone chips in PFTBA (3D analysis); (Red=new bone formation; White=bone chips); and

FIGS. 8A and 8B show histological analysis of bone growth in mice 21 days after implantation of 8A) DBM and bone chips in PBS; 8B) DBM and bone chips in PFTBA.

### DETAILED DESCRIPTION

An improved composition for inducing bone growth is provided that is a combination of at least DBM and an oxygen carrier. Implantation of a composition of DBM and an oxygen carrier results in enhancement of bone formation compared to DBM alone. That is, after intramuscular implantation, bone formation was found to be greater after injection of a composition of the present invention comprising DBM and an oxygen carrier (e.g. a perfluorocarbon) than a composition of DBM alone (in PBS).

DBM of various forms which are suitable for implantation can be used in combination with an oxygen carrier. The various forms of commercially available DBM include putty, gel, strips, paste, sheets, circular grafts, fibers, and matrices. The amount of DBM to be used ranges from approximately 0.5 ml (cubic centimeters, cc) to approximately 10 mls (ccs) depending on the site of the subject requiring bone formation. The form of DBM to use depends on the application, as will be apparent to one skilled in the art. Methodologies and uses of the various forms of DBM are disclosed in the following: Martin et al., Spine, 24:637-645, 1999; Khan et al., J. Am Acad. Orthop. Surg., 13: 12-137, 2005; Peterson et al., J of Bone and Joint Surg., 86-A, No. 10, October 2004; Sassard et al., Orthopedics, 23:1059-1064, 2000; Louis-Ugbo et al., Spine, 29:360-366, discussion Z1, 2004; Cammisa et al., Spine, 29:660-666, 2004.

Examples of oxygen carriers include, but are not limited to, perfluorocarbon-based oxygen carriers such as perfluorotributylamine [PFTBA; (C₄F₉)₃N], perfluorooctylbromide [PFOB; C₈F₁₇Br] (Khattak, S. F. et al., Biotechnol. Bioeng. 96: 156-166, 2007), and perfluoro-n-octaine (Perfluoron®). Additional examples of perfluorocarbon-based oxygen carriers include, but are not limited to, octafluoropropane, perfluorohexane, perfluorodecalin, perfluorodichlorooctane, perfluorodecane, perfluorotripropylamine, perfluorotrimethylcyclohexane, perfluoroperhydrophenanthrene, perfluoromethyladamantane, perfluorodimethyladamantane, perfluoromethyldecaline, perfluorofluorene, diphenyldimethylsiloxane, hydrogen-rich monohydroperfluorooctane, alumina-treated perfluorooctane, and mixtures thereof. Oxygen carrier refers to a molecule capable of transporting, delivering and/or supplying oxygen to impart viability, proliferation, and differentiation to surrounding cells.

In one embodiment, the amount of oxygen carrier in the DBM composition ranges from approximately 5% to approximately 60% (w/v) (Kimelman-Bleich et al., Biomaterials, 30:4639-4648, 2009; Keipert, In: Art. Cells Blood Subst. lmmob Biotech, 23, 281-394, 1995; Keipert, Blood Substitutes, R. W. Winslow, Academic Press, London, p. 312, 2005). In one embodiment, PFTBA is used as the oxygen carrier in a range of approximately 5 to 20% (w/v) with DBM. In one embodiment, Perfluoron® (Alcon Laboratories Inc., Fort Worth, Tex., USA) containing perfluoro-n-octane, is used at the oxygen carrier. In one embodiment, the oxygen carrier is a composition of perfluorohexyloctane and silicone oil polydimethylsiloxane 5 (F6H8S5) (Novaliq GmbH, Heidelberg, Germany) (Brandhorst et al., 2010, Transplantation, 89:155-160). The amount of oxygen carrier can vary depending on the specific oxygen carrier used (Gomes and Gomes, “Perfluorocarbon Compounds Used As Oxygen Carriers: From Liquid Ventilation to Blood Substitutes,” 2007).

The composition and method of the present invention may be applied to any subject having a condition that requires or would be improved with enhanced or induced bone formation.

Subjects that may require bone formation by administration of the composition of the present invention include animals, such as humans, in need of bone growth.

The term “implanting” refers to administering the composition of the present invention by methods known in the art. Known methodologies for implanting are disclosed, for example, see Martin et al, Spine, 24:637-645, 1999; Khan et al., J. Am Acad. Orthop. Surg., 13: 12-137, 2005; Peterson et al., J of Bone and Joint Surg., 86-A, No. 10, October 2004; Sassard et al., Orthopedics, 23:1059-1064, 2000; Louis-Ugbo et al., Spine, 29:360-366, discussion Z1, 2004; Cammisa et al., Spine, 29:660-666, 2004.

The DBM and oxygen carrier composition of the present invention may be supplemented with at least one of the following: bone chips (autologous or allograft), growth factors, fibrin, collagen, synthetic scaffolds, and bone marrow-derived stem cells (e.g. hematopoietic, stromal, and mesenchymal stem cells).

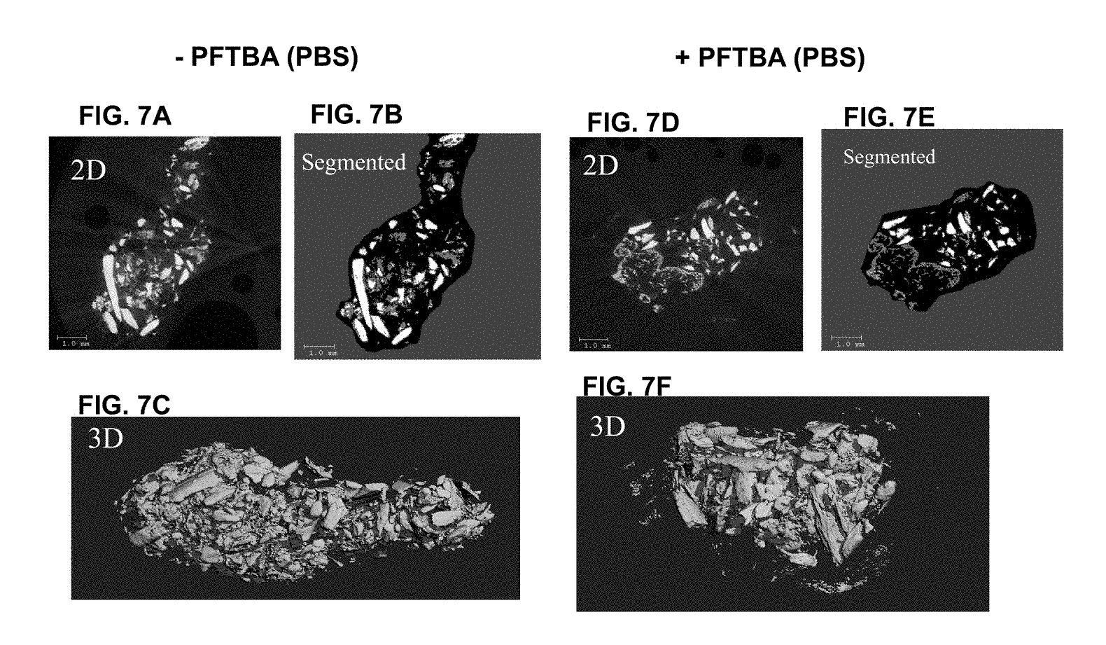

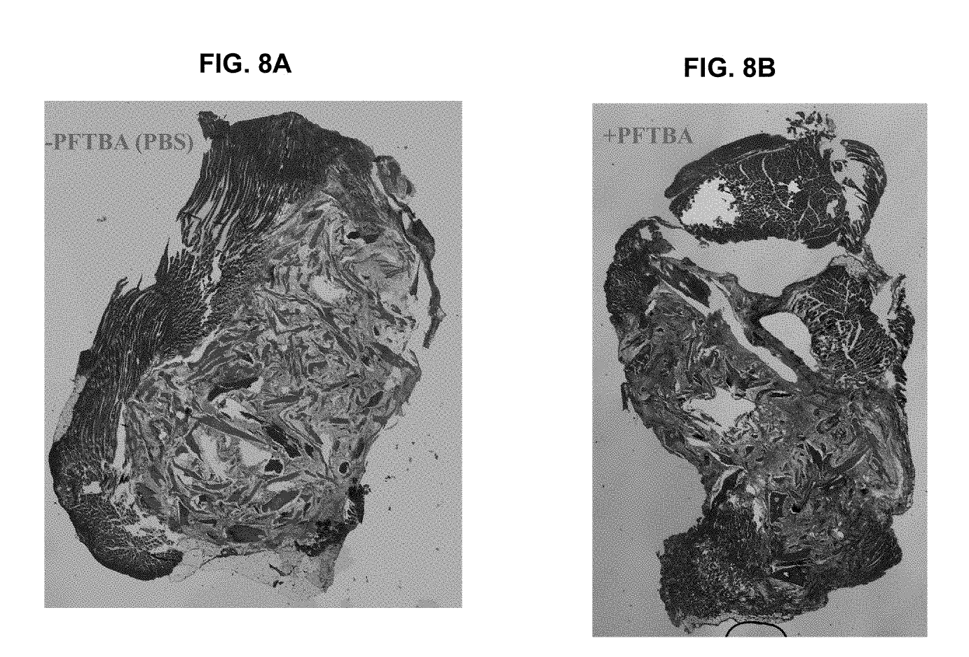

As shown in FIGS. 7A-7F and 8A-8B and detailed in Example 2, autologous bone chips were added to the DBM +/−PFTBA emulsion.

Growth factors, such as those in the transforming growth factor beta (TGFI3) superfamily, are known for their ability to induce bone formation in ectopic and orthotropic sites. Members of the TGF(3 superfamily include BMP-2, BMP-6, BMP-7, and BMP-9, which have been shown to induce osteogenic differentiation (Kang et al., 2004, Gene Ther., 11:1312-1320).

Methods for the addition of fibrin, collagen, synthetic scaffolds, and bone marrow-derived stem cells are known in the art and described in US 2009/0214649 of which paragraphs 0072-0082; 0100-0111; and 0168 are herein incorporated by reference.

### EXAMPLE 1

DBM in PFTBA. 600 μl of Grafton® DBM putty was mixed in an Eppi tube with 180 μl of PFTBA (Sigma-Aldrich) or PBS to form an emulsion of 10% PFTBA weight/volume or 10% PBS weight/volume (10 g/ml). For every ml (milliliter) of DBM/PFTBA emulsion, 90 mg lecithin E80 (Lipoid GmbH, Ludwigshafen, Germany) was added to 330 μl PFTBA and 660 μl PBS. This solution was sonified at 10% amplitude for 90 seconds (Branson Sonifier 450 Model 1020 probe sonicator, Danbury, Conn., USA). For the DBM/PBS emulsion, 990 μl PBS was emulsified with 90 mg lecithin E80. 100 μl of the DBM/PFTBA or DBM/PBS emulsion was then implanted by syringe intramuscularly into NOD/SCID (immunodeficient) mice, as described (US 2009/0214649). 21 days post implantation, the implant region was harvested and bone formation was analyzed using micro-computed tomography (micro-CT or μCT) and histological staining. Histological staining can be carried out following methods known in the art. See for example, Sheyn et al., Gene Ther., 15: 257-266, 2008.

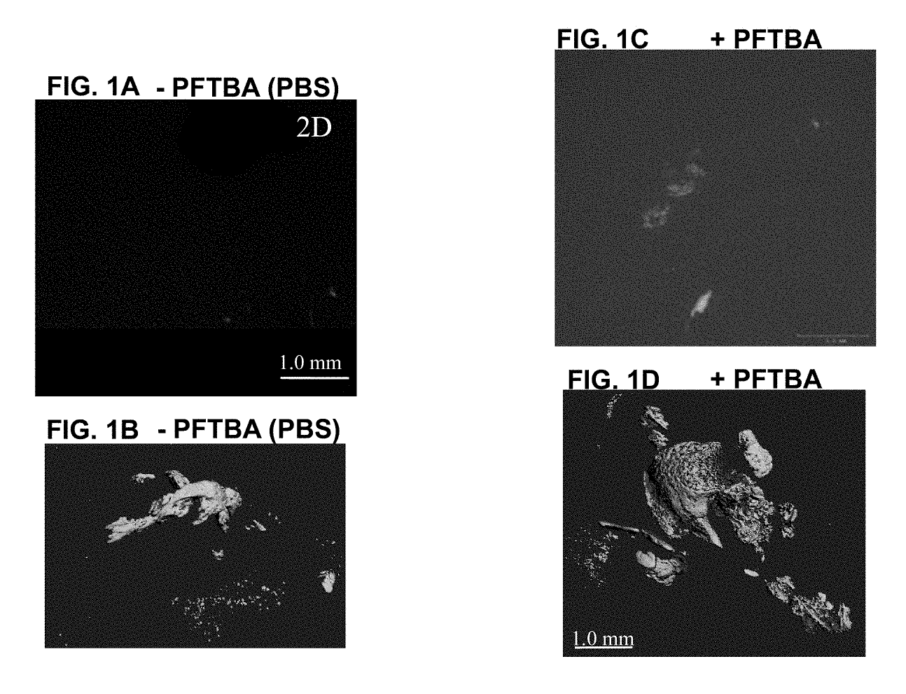

FIGS. 1A-1D show 2D and 3D micro-CT images of bone formation 21 days after implant. FIGS. 1C, 1D (DBM with PFTBA) show a higher volume of new bone than FIGS. 1A, 1B (DBM in PBS).

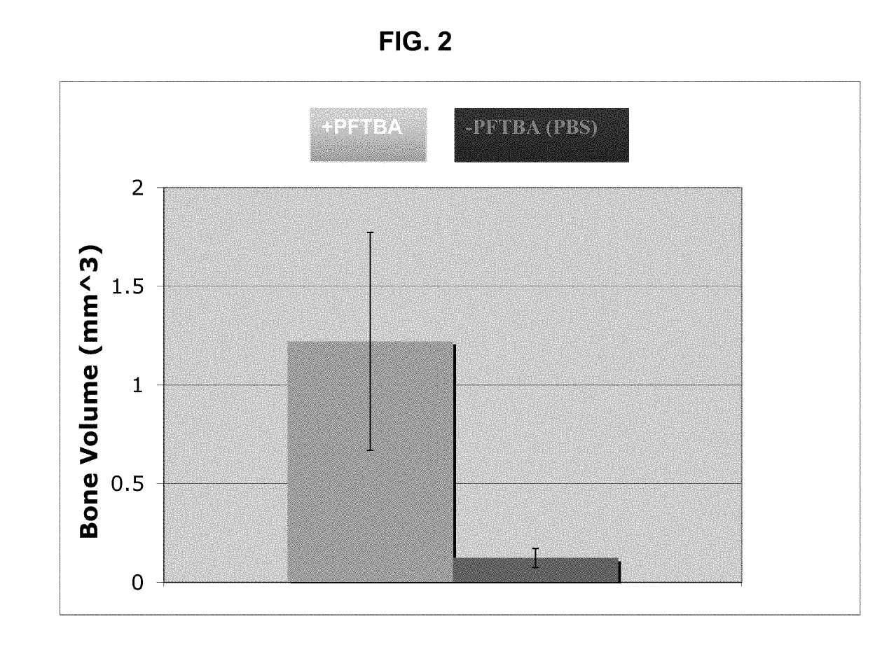

The histogram of FIG. 2 represents bone volume analysis in five samples. FIG. 2 shows that a significantly higher volume (mm³) of new bone (an approximate 10-fold increase in bone formation) was detected in DBM implants supplemented with PFTBA (left blue bar) than DBM in PBS (right red bar) with P<0.05, Student's T-test, n=5.

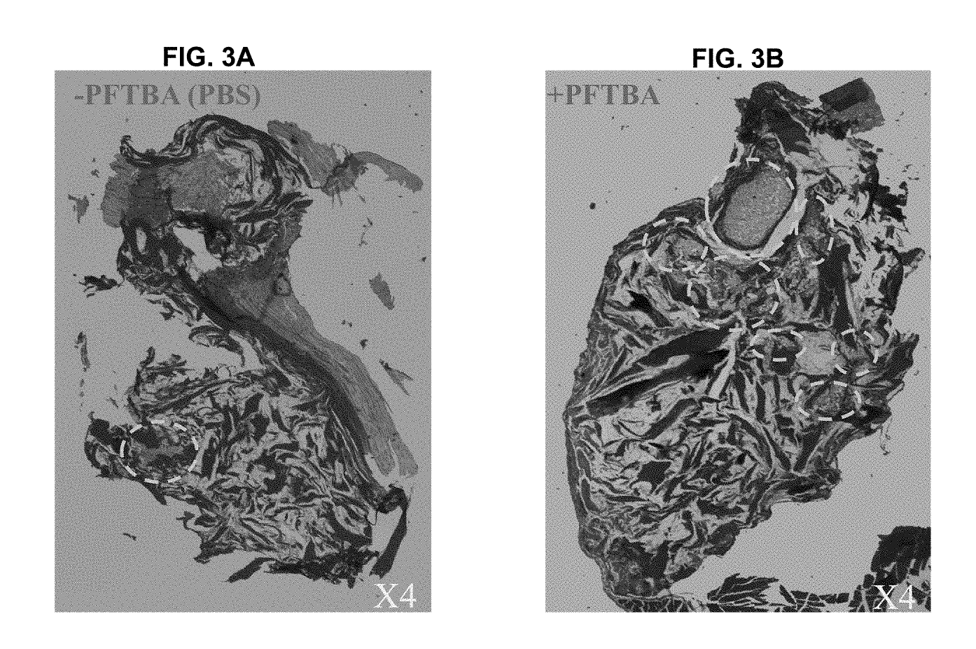

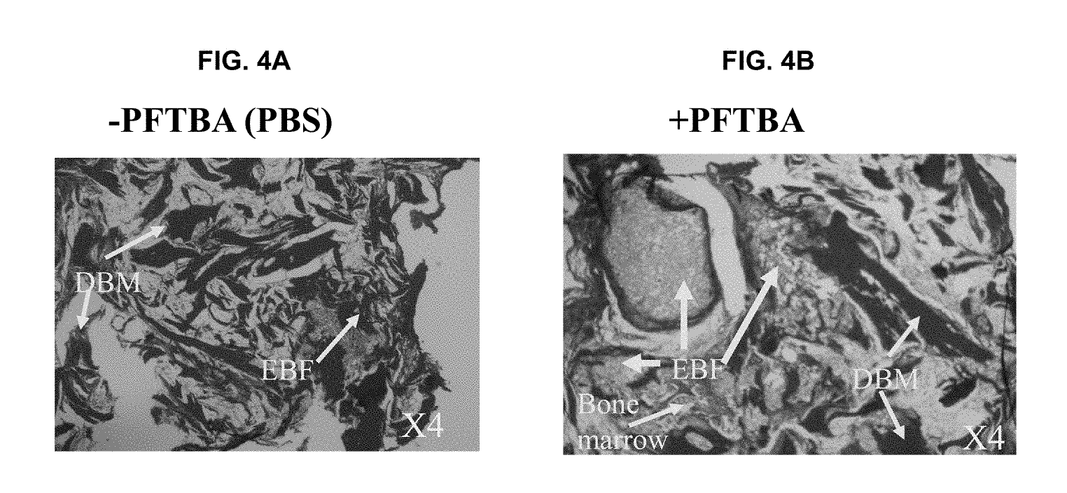

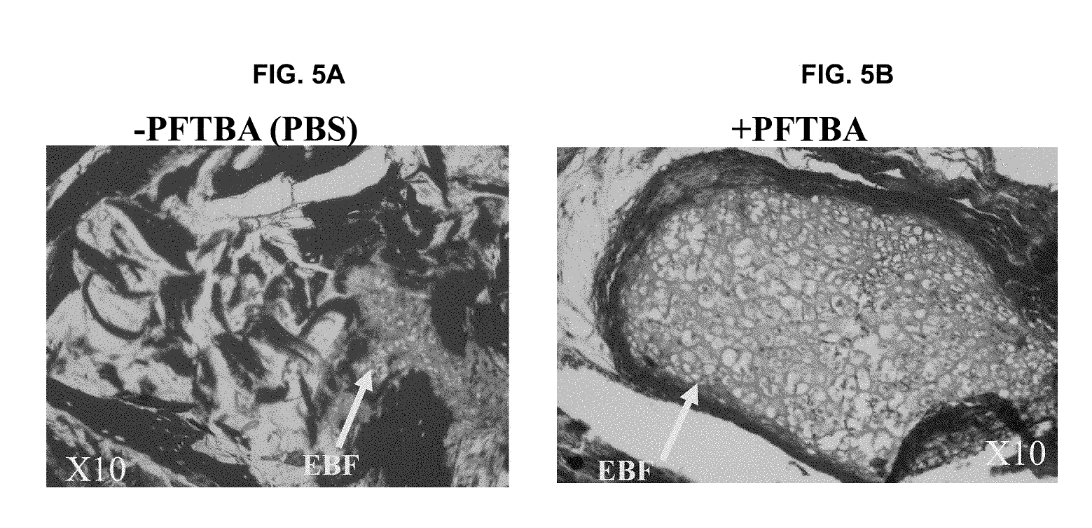

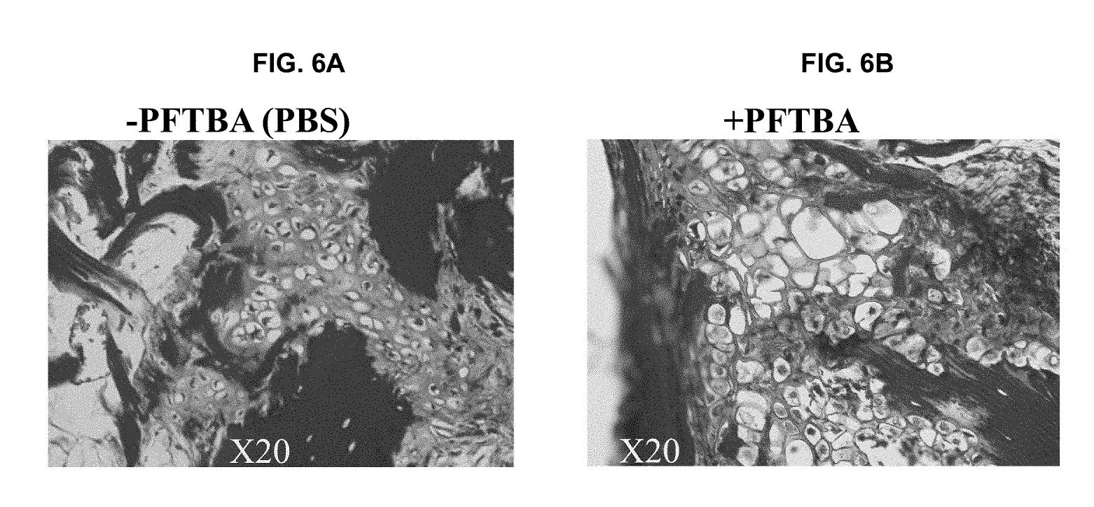

Histological analysis of the harvested DBM/PBS and DBM/PFTBA implants are shown in FIGS. 3A-3B, 4A-4B, 5A-5B, and 6A-6B, at X4, X10 or X20 magnification as shown. Digitated circles are drawn around endochondral bone formation (EBF), and DBM is labeled as well as bone marrow.

### EXAMPLE 2

DBM and Bone Chips in PFTBA. 600 μl of Grafton DBM putty was mixed with 300 μl of harvested and ground bone chips, to which 300 μl of PFTBA (or PBS) was added to form an emulsion of 10% PFTBA weight/volume (10 g/ml). Implantation was carried out as above using 100 μl of the DBM/Bone Chips +/−PFTBA in NOD/SCID mice.

FIGS. 7A-7D show 2D, segmented, and 3D micro-CT images of bone formation 21 days after implant with DBM and bone chips in PBS (FIG. 7A-7C) or in PFTBA (FIG. 7D-7F).

Histological analysis of the harvested DBM/Bone Chips/PBS and DBM/Bone Chips/PFTBA implants are shown in FIGS. 8A-8B.

In summary, a composition and method for inducing bone growth are provided. Bone growth is induced (or enhanced) upon implantation of DBM and an oxygen carrier compared to DBM in PBS. While the present invention has been illustrated and described with reference to certain exemplary embodiments, those of skill in the art will understand that various modifications and changes may be made to the described embodiments without departing from the spirit and scope of the present invention, as defined in the following claims.

What is claimed is:

1. A method of inducing bone growth, comprising:
implanting an effective amount of a composition for implantation within a patient for inducing bone growth at a target site in the patient, the composition consisting of:
a perfluorocarbon in an amount of about 5% to about 60% (w/v);
an emulsifier; and
demineralized bone matrix (DBM),
wherein the composition is free of cultured cells and is capable of inducing bone growth in the patient in the absence of cultured cells and the composition optionally consists of autologous bone chips, allograft bone chips, growth factors, fibrin, or collagen.

2. The method of claim 1, wherein the perfluorocarbon is selected from the group consisting of perfluorobutylamine (PFTBA), perfluorooctylbromide (PFOB), perfluoro-n-octane, octafluoropropane, perfluorohexane, perfluorodecalin, perfluorodichlorooctane, perfluorodecane, perfluorotripropylamine, perfluorotrimethylcyclohexane, perfluoroperhydrophenanthrene, perfluoromethyladamantane, perfluorodimethyladamantane, perfluoromethyldecaline, perfluorofluorene, diphenyldimethylsiloxane, hydrogen-rich monohydroperfluorooctane, alumina-treated perfluorooctane, and mixtures thereof.

3. The method of claim 1, wherein the perfluorocarbon is perfluorodecalin.

4. The method of claim 1, wherein DBM is in the form of putty, gel, strips, paste, sheets, or fibers.

5. The method of claim 1, wherein the DBM is in the form of fibers.

6. The method of claim 1, where the emulsifier is lecithin.

7. A method of inducing bone growth, consisting of:
implanting an effective amount of a composition for implantation within a patient for inducing bone growth at a target site in the patient, the composition consisting of:
a perfluorocarbon in an amount of about 5% to about 60% (w/v);
an emulsifier; and
demineralized bone matrix (DBM),
wherein the composition is free of cultured cells and is capable of inducing bone growth in the patient in the absence of cultured cells and the composition optionally consists of autologous bone chips, allograft bone chips, growth factors, fibrin, or collagen.

8. The method of claim 7, wherein the perfluorocarbon is perfluorodecalin.

9. The method of claim 7, wherein DBM is in the form of putty, gel, strips, paste, sheets, or fibers.

10. The method of claim 7, wherein the DBM is in the form of fibers.

11. The method of claim 7, where the emulsifier is lecithin.
